(19) United States
(12) Patent Application Publication
(10) Pub. No.: US 2003/0103996 A1
(43) Pub. Date: Jun. 5, 2003

(54) Cell-based vaccine

(76) Inventors: Martha Eibl, Vienna (AT); Thomas Kreil, Klosterneuburg (AT); Josef Mannhalter, Vienna (AT); Johann Eibl, Vienna (AT); Astrid Kerschbaum, Vienna (AT); Peter Bruhl, Vienna (AT)

Correspondence Address: BAKER & BOTTS, 30 ROCKEFELLER PLAZA, NEW YORK, NY, 10112

(21) Appl. No.: 10/255,423

(22) Filed: Sep. 26, 2002

Related U.S. Application Data
(63) Continuation of application No. 09/224,807, filed on Dec. 31, 1998, now abandoned.
Application No. 09/224,807 is a continuation of application No. PCT/EP97/03452, filed on Jul. 2, 1997.

(30) Foreign Application Priority Data
Jul. 2, 1996 (DE) 196 26 614.9

(51) Int. Cl. A61K 39/12; A61K 39/00; C12Q 1/68

(52) U.S. Cl. 424/185.1; 424/204.1; 424/192.1; 424/186.1; 435/6

(57) ABSTRACT

The invention relates to a complex comprising an antigen of a microbial or molecular pathogen and mammalian cell constituents in isolated or purified form. A vaccine composition containing the complex, a corresponding pharmaceutical preparation and a method of preparing the complex are also disclosed.

The present invention relates to a complex comprising an antigen of a pathogen and a cell constituent. Furthermore, a vaccine composition containing said complex, a suitable pharmaceutical preparation as well as a process for preparing said complex are provided.

Microbes such as viruses, bacteria, fungi or parasites may infect a plurality of host organisms, cause pathologic damage and, in the event of uncontrolled propagation, even kill their host. Thanks to the immune system, pathogens are recognized and efficiently combated.

In recent years, prions have been discussed as new infectious pathogens of the molecular type. Prusiner S. et al., Advances in Virus Research, Vol. 29, pp. 1-56, 1984, have described diseases ascribed to prions, among them scrapie, kuru, Creutzfeldt-Jakob disease, Gerstmann-Straussler-Scheniker syndrome and familial insomnia. Although prions are attributed molecular structures, nucleic acids which are responsible for the expression of the prions can not be excluded. The cultivation of prions in vitro, or the infection of cell lines for obtaining the respective titer has been described by Race R. et al., Current Topics in Microbiology and Immunology, Vol 172, pp. 181-193.

The treatment of patients exposed to risks of infection on account of prions or “slow viruses” has gained increasing importance. The animal models and isolated molecules available to this end may be employed for testing appropriate treatment strategies.

In terms of function, the immune system consists of native, relatively unspecific resistance mechanisms, on the one hand, and of acquired, very specific immunity effector functions on the other hand. In terms of immunity effector mechanism, it is, moreover, to be differentiated between humoral immune response, i.e., the formation of antibodies against antigens of a pathogen, and cellular immune response.

Immunity against an infectious pathogen may be generated by active or passive immunization. In passive immunization, the humoral or the cellular protection of an organism which has built up that protection is transferred to another naive organism. The effect occurs immediately, yet the duration of protection is limited.

Active immunity nay be induced by infection with a pathogens or it may be achieved by administering a vaccine. In both of these procedures, the immune system is activated, e.g. inducing the proliferation of antigen-reactive T-cells or B-cells, from which memory cells are formed subsequently. Active immunity will therefore, be maintained over a long period of time, even for years, and may be renewed by a booster.

A number of vaccines have been available for active immunization against a plurality of infectious pathogens, the design of such vaccines being manifold.

At present, the following types of vaccines have been used in the main: whole-virus vaccines (in the form of inactivated, killed virus or attenuated live virus vaccines), purified or recombinant vaccines comprised of subunits of a pathogen and referred to as subunit vaccines, recombinant vector vaccines, synthetic peptide vaccines or anti-idiotype vaccines. More recent developments in the production of vaccines have increasingly concentrated on the use of “naked” nucleic acids (e.g., DNA vaccines).

Depending on the design of a vaccine and its out of administration a different type and a different extent of the immune reaction is to be expected. A frequently encountered problem in vaccination is that the administered vaccines are little immunogenic and not even multiple administrations will result in protective immunity.

For the above reasons, so-called immunostimulants or adjuvants are frequently admixed to the vaccines. Such substances bring about an intensification of the immune response (inmmunopotentiators), or they influence the type of immune response (immunomodulators). For that purpose, inorganic substances such as aluminum hydroxide as well as the use of water-in-oil emulsions with mycobacteria (complete Freund's adjuvant) and without mycobacteria (incomplete Freund's adjuvant) have been known for long in the prior art.

Various cytokines such as, e,g., interleukins or lymphokines also possess adjuvanting properties.

When choosing an adjuvant or a suitable combination of antigen and adjuvant, it is to be taken into account that not every adjuvant will enhance the immune response to any antigen and that the choice of antigen may also influence the type of the immune response induced. It has, therefore, been of continued interest to find substances having adjuvanting properties for vaccines.

From a more recent work (Heike M. et al., 1994, J. Immunotherapy 15: 165-174), the in vitro solution of cytotoxic T cells by plasma membranes of mouse fibroblasts is, for instance, known. That document also describes the induction of antigen-specific MHC class I restricted cytotoxic T cells determined after the immunization of mice with syngeneic membranes of UV-induced tumor cells, SV40 transformed fibroblasts or non-productively influenza-infected fibroblasts. These membranes are administered to mice in order to induce a cellular immune response.

It is known also from Bachmann M. F. et al., 1994, Eur. J. Immunology 24, 2128-2236 that cellular fragments of insect cells may be used as adjuvant for a recombinant antigen in order to induce cytotoxic T cells. The antigen and the membrane or a membrane fragment may be administered as a mixture.

In addition to the induction of cellular immunity, also the induction of a humoral immune response is desirable in most cases. Although the importance of humoral and cellular immunities differs from pathogen to pathogen, both will usually contribute to the success of an immune response.

Peiris J. S. and Porterfield J. S., 1979, Nature 282, 509-511, have shown that the presence of antibodies to a virus particle may be associated with a more efficient virus infection (“antibody-dependent enhancement of viral infectivity”). Antibodies against a virus, therefore, also may contribute to a deterioration of clinical prognosis, and attempts have been made to avoid a problem of this kind when developing a vaccine.

Another problem, potentially associated with so-called “subunit” vaccines, is that the administered antigen is little immunogenic, which may be related to an insufficient presentation of the antigen to immunocompetent cells.

It is, therefore, an objective of the present invention to provide a highly effective immunogen while avoiding the disadvantages known from prior art. Among other things, the objective is to present the antigenic determinant in a manner which will achieve protective immunity, i.e., sufficient protection will be available against a pathogen in the event infection with said pathogen occurs.

In accordance with the invention, this objective is achieved by the subject matter of the present patent claims.

The subject matter of claim 1, thus, is an antigen of a microbial or molecular pathogen complexed with a cell constituent in the isolated or purified form, wherein the cell constituent is derived from mammalian cells. Preferred mammalian cells are those infected with the pathogen and expressing the antigen.

Surprisingly, it has been found that the complex according to the invention possesses an excellent ability to induce protective immunity, expressed by a humoral and optionally cellular immune response.

It is the very association of antigen and cell constituent which in curtain cases on the one hand is perfectly to present the respective antigen and on the other hand will also allow co-stimulating signals on account of the cell constituent to enter into effect upon administration of the complex, e.g. to a mammal.

The cell constituent of the complex according to the invention is derived from an animal or human cell or is a synthetic equivalent. Thus, constituents of prokaryotes or insects are avoided in a respective preparation for human use.

Autologous, homologous or heterologous cells may be used as starting materials for the cell constituent of the complex according to the invention. Cells exhibiting the capacity of a competent antigen presentation are preferably chosen. Also erythroctes, leucocytes, thrombocytes, lymphocytes, thymocytes, granulocytes, monocytes, primary cells or cell lines may be used as cell constituent or for its production.

Examples of mammalian cell lines comprise cell lines suitable for the preparation of vaccines, such as Vero cells, CHO or HeLa cells, but also align or immortalized cells and, in particular, tumor cells are suitable to be used according to the invention.

The cell constituents used preferably have co-stimulating effects, rendering cell activation possible. In a preferred embodiment, the cell constituents are derived from a stimulated or activated cell. Such a cell may be obtained by exposing it to special stimuli. Thus, mitogens, hormones, cytokines or other cells may be added in order to provoke a reaction or alteration of the cell. Preferably, the cell constituents are not derived from resting cells and, in particular, resting fibroblasts.

The cell constituent of the complex according to the invention preferably is derived from infected cells and, in particular, from cells infected with a pathogen, according to a preferred embodiment, the cell constituent is derived from a whole-virus-producing cell or from a cell replicating viral constituents. It is, thus, no latently infected cell.

In a preferred embodiment, the cell constituent of the complex according to the invention is obtained directly from a cell infected with a pathogen. To this end, the cell, preferably a cell line suitable for the production of human vaccines is infected with the pathogen e.g. a virus, most preferably a human pathogenic virus, according to conventional methods. Thereafter, a cell constituent is recovered, for instance a membrane fraction of the virus-infected cell to which antigens of the pathogen have been bound or will be bound, which per se are not present in the unbound mature virus. In particular, these are so-called non-structural proteins of a virus.

Preferably, the cell constituent is obtained in a fraction containing the cell membrane or an intracellular structure of the cell. The cell constituent may, however, also be the cell as such. In the case of a membrane or a membrane fragment constituting the cell constituent, this may be a plasma membrane or a membrane of cell organelles. As cell organelles, the endoplasmatic reticulum, the Golgi apparants, lysosomes and others may be cited.

In accordance with the invention, the cell constituent also may constitute an optionally synthetic equivalent of a membrane or a membrane fragment having a similar structure and an identical function as in the complex according to the invention. According to a further embodiment, this constituent or its respective equivalent may also be produced synthetically, e.g. by chemical methods.

The protein portions of the membrane may also be produced in a recombinant manner and assembled with lipids to form a membrane. Synthetic liposomes or mixtures of different synthetically produced or naturally occurring lipids or phospholipids may also be used as membranes.

Mixtures of the above-specified membranes membrane fragments or cell structures are also suitable for the preparation of the complex according to the invention.

In another preferred embodiment, the cell constituent is derived from cells originating from the species for which the virus would be pathogenic. For instance, the virus is a human-pathogenic virus and the cell constituent is derived from a human cell.

In a further preferred embodiment, the cell constituent is a substance selected from the group consisting of membrane protein, intracellular protein, factor derived from activated lymphocytes and activator for lymphocytes or a combination thereof, and the cell fraction contains such a substance or a combination of such substances. Such substances are obtainable from a cell fraction or are used in purified form. The membrane protein or the intracellular protein preferably is an alloantigen, chemoline receptor, chemoline or lymphokine. Any combinations thereof are also contemplated by the invention. The alloantigen, in particular, is an MHC class I or MHC class II antigen. Thus, the heat shock protein should be exemplified as an intracellular protein. An example of a factor derived from activated lymphocytes is a factor capable of suppressing viral growth. According to the invention, activator for lymphocytes serves to denote a substance capable of, e.g., inducing one of the aforementioned factors.

For example, the cell constituent may be obtained after the selective transformation, transfection or activation of an appropriate cell. The previously mentioned substances may, however, also be admixed to the cells as additives in defined quantities.

In a further preferred embodiment, the cell constituent is derived from a cell containing a recombinant gene. This renders possible the use of an antigen encoded by a nucleic acid transferred into a cell and expressed by the cell, of the pathogen. To this end, genetic engineering methods or recombinant nucleic acids, i.e., recombinant DNA technologies are used.

Antigens encoded by the genome of a pathogen are expressed in a host cell of this type. The nucleic acid of the pathogen or parts thereof may be introduced into the genome of the host cell via a suitable vector such as recombinant viruses or plasmids, or directly, such that the transformed or transfected cell expresses the desired antigens of the pathogen.

A number of methods are available to these technologies. The construction of the respective vectors preferably encompasses suitable promoter elements such as, e.g., SV40, CMV, RSV, LTR, EBV, β-actin, hGH T4, T7, SP6, metallothionein, adeno2, adeno major late, or TK promoters or muscle-specific promoters such as myosin promoters or inducible promoters such as hsp- or β-interferon promoters. Examples of appropriate DNA expression vector systems include pBPV, pSVL, pRclCMV, pRc/RSV, myogenic vector systems (WO 93/09236) or vectors of viral systems such as pox viruses (U.S. Pat. No. 5,445,953), adenoviruses, retroviruses, baciuloviruses, herpes viruses and polio viruses.

The antigen encoding expression vector is then used for transforming the host cell. The host cell is an eukaryotic host cell. Preferred mammalian cells are CHO, COS, BHK, SK-HEP, C127, MRC5, 293, Vero cells, fibroblasts, keralinocytes or myoblasts, hepatocytes or parent cells.

The respective antigen preferably is a protective antigen or an antigen acting protectively by association with the cell constituent. The term protection implies protection against an infectious disease, which protection may be demonstrated using a suitable animal model or surrogate marker such as a defined antibody titer.

Protective antigens of a pathogen, in particular, are considered to be peptides, polypeptides or proteins derived from the genome of a pathogen, the protective effect of which may be detected in the complex according to the invention; a respective nucleic acid of the pathogen may, however, be used as well. Preferably, the pathogen is a virus, most preferably selected from the group consisting of flaviviruses, herpes viruses, hepatitis viruses, retroviruses, influenza viruses, coxsackie virus, tumor viruses and echo viruses.

Selected viruses are from the family of Flaviviridae, preferably, encephalitis virus such as TBE virus and, in particular, TBE virus of the western sub-type, or from the subgroup of Dengue viruses. Other selected viruses are from the family of Hepadnaviride and, preferably, hepatitis A, B, C, D or X virus. Further selected viruses are from the family of Orthomyxoviridae and, preferably, influenza viruses. From the family of Piconiaviridae, viruses from the genus of the enietoviruses such as, e.g., coxsackie virus, or from that of rhinoviruses are preferably selected. From the family of Retroviridae, an HI virus and, in particular, HIV-1 and HIV-2 are preferably selected.

Antigens from bacterial pathogens preferably are derived from E. coli, Bordetella, Botrelia, Pseudomonas, Haemophilus, mycobacteria, streptococci, salmonellae, Helicobacter and clostridiae. Antigens originating from parasites preferably are selected from the group consisting of Amaebida, Trypanosoma and Plasmodium. Antigens originating from molecular pathogens preferably are derived from the causative agents of the Creutzfeldt-Jakob disease, scrapie, kuru and BSE.

If the complex according to the invention in a preferred embodiment is obtained by infection of a cell with a virus, the antigen, or the complex, may be obtained in an early phase of virus replication. In addition, it is also possible to obtain the antigen, or the complex, in the late phase of virus replication. Depending on the replication stage, it is possible to obtain certain antigens or cell constituents which are present in the resting cell or in the whole virus not at all or not in that form.

An example of a protein having different phenotypes during virus replication is the preM protein, which occurs in the development of a Flaviviridae virus and is no longer present in the mature virus particle, having been completely processed to M protein. Examples of virus-encoded proteins not occurring in the free virus particle are, for instance, NS1, NS3, NS5 or other non-structural proteins of Flaviviridae.

In the complex according to the invention, the antigen may form several epitopes and even constitute a whole pathogen, e.g., a whole virus and, in particular, an inactivated or attenuated virus, or part of a pathogen, e.g., a subviral particle.

In a preferred embodiment, the complex according to the invention comprises an antigen composition responsible of protectivity and, in particular, at least one non-structural protein of the pathogen. The non-structural protein for instance, is a regulatory protein, a protein having an enzymatic function such as reverse transcriptase, RNA polymerase, integrase or protease, or a protein having no function or a yet unknown function. Also the respective precursor molecules of these proteins or structural proteins as well as the respective derivatives with comparable or even enhanced imnmunogenic action such as (chemically) modified proteins, fragments, fusion proteins, mutants, analogs and the like may be used according to the invention. The mutants preferably comprise an amino acid sequence having at least 80% homology with the corresponding native protein.

In case the complex according to the invention comprises a non-structural protein of a pathogen, it is possible, when used as a vaccine, to obtain immunization with antigens, which cannot be obtained when using common subunit vaccines or vaccines based on whole pathogens. This is an advantage, in particular, in systems where an antibody-dependent enhancement of infection is to be feared on account of antibodies against structural proteins of a virus. Immunity against a non-structural constituent of a virus renders possible a successful immune response without that drawback.

Other preferred antigens are comprised of several subunits. These complex antigens, e.g., consist of an envelope protein and/or a nucleoprotein of the pathogen and a non-structural constituent of the pathogen. Preferably, at least one non-structural protein and a structural protein are present as a complex antigen.

The complex linkage in the complex according to the invention may be a covalent linkage, optionally by means of a linker. A covalent linkage is also feasible by chemical cross linking. The simple formation of sulfur bridges likewise constitutes a kind of covalent-linkage. The complex linkage may, however, also be based on electrostatic, hydrophobic or van der Waals' forces. In another preferred embodiment, the antigen and the cell constituent are adsorbed or coadsorbed on a solid carrier. The solid carrier may also be a lipid constituent such as a liposome or a phospholipid vesicle.

The linkage of the antigen to the cell constituent in the complex according to the invention surprisingly continues to exist even if one of the two constituents or both of the constituents are treated further. Treatment may be effected e.g. for the purpose of inactivation, such as by sonication, irradiation, chemical treatment, heat treatment or enzyme treatment. Preferably, a chemical formalin treatment is carried out such that the pathogen will no longer be infectious or act as a pathogen after administration.

According to the invention, the cell may be used as a starting rate for the production of the complex according to the invention, either untreated or in a modified form. It may be subjected to lysis or may be present as an intact cell. Thus, in order to prepare a cell fraction, the cells are, e.g., lysed, the cell lysate is fractionated, optionally by density gradient centrifugation, and the respective fraction is isolated.

In another preferred embodiment, the complex according to the invention is present in the purified form. That purification may be effected by various methods of purification for high-molecular substances. Such a purification may e.g. be effected as a function of the size or charge of the substance. An immunogenic complex according to the invention may be purified by centrifugation (e.g.; density gradient centrifugation), dialysis/diafiltration, chemical precipitation or by means of chromatography, in particular, by gel filtration, ion exchange, hydrophobic chromatography, affinity chromatography or reversed phase chromatography. Also a combination of purification methods may be contemplated for obtaining a highly purified immunogen. Free antigens, i.e., antigens not associated with cell constituents are separated by purification. Preferably, existing nuclei are eliminated. Free cell constituents are separated as well. The complex according to the invention is characterized in its purified form to the extent that further purification will not result in a substantial separation of free antigen or cell constituent.

Antigen and cell constituent may be purified also independent of each other, the association to form the complex in that case being effected are or during purification.

The complex according to the invention may be present as a preparation additionally containing a cell constituent such as a membrane, preferably derived from non-infected, non-transfected or non-transformed cells, in an immunogen-enhancing amount, i.e., as an adjuvant. That preparation is suitable primarily for immunizing mammals and, in particular, primates or human beings.

According to a preferred embodiment, the complex according to the invention contains an inactive virus as an antigen. Preferably, the virus is an inactivated virus and, in particular, a virus inactivated by a chemical and/or physical process. Such a chemical process may comprise formaldehyde treatment. A physical process for inactivation may consist in heat, irradiation or ultrasonic treatment.

In a particularly preferred embodiment, the complex according to the invention is comprised of an inactive virus having incorporated a cell constituent, as is the case in the so-called virus budding process. In particular, it has incorporated a cell constituent containing, or enriched with, substances selected from the group consisting of membrane protein, intracellular protein, factor derived from activated lymphocytes and activator for lymphocytes.

According to a preferred embodiment, the mammalian cell or cell fraction is enriched with at least one constituent according to any one of claims 9, 14 or 15.

Enrichment may be effected e.g. by admixing the desired constituent to the cell or cell fraction, by inducing the formation of the desired substance, e.g. by adding chemokines or interferons, such as interferon-gainma, or by a molecular biological gene transfer method such as retroviral gene transfer or transfection of the cell with an appropriate gene.

As already mentioned earlier, the pathogen with which the cell is infected may be a virus and, in particular, an inactive virus. In case the cell or cell fraction is enriched with the described constituents, a complex comprised of a virus enriched with those constituents may then be obtained.

The invention also provides a vaccine composition which contains the complex according to the invention in a pharmaceutically acceptable form, and optionally additional antigens or adjuvants. Additional adjuvants comprise current substances known from the prior art. Thus, various cytokines may be employed. Furthermore, inorganic substances such as aluminum or iron compounds, including the corresponding hydroxides, are also used.

The vaccine composition according to the invention preferably is present in an amount suitable for the therapeutic or prophylactic treatment of mammals and, in particular; primates including man, bearing the risk of the disease caused by the pathogen.

The vaccine composition according to the invention is suitable, e.g., for treating infections or infectious diseases caused by HI viruses such as AIDS, various disorders of the nervous system, such as encephalitis, for treating infections with influenza viruses, autoimmune diseases etc.

An advantage of the complex according to the invention resides in the fact that the complex-bound antigen avoids the problem of infection enhancement. Immunization using the complex according to the invention may, in fact, result in immunity against the infected cells rather than against the free pathogen. Thus, the suitable choice of cell constituent and a non-structural constituent of a pathogen will especially induce immunity against the non-structural constituent of the pathogen which is directed against the pathogen-infected cell but does not result in an antibody-dependent enhancement of infection.

Furthermore, the action of the vaccine according to the invention may be universal and directed against several subtypes of a virus such as Dengue viruses, or against several members of the same virus family such as several flaviviruses e.g., by inducing immunity against preserved non-structural proteins, especially those having regulatory functions.

The vaccine composition according to the invention may be available in a form suitable for parenteral administration or for the stimulation of mucosal immunity. Thus, it may be present in a form suitable for intravenous intramuscular, transdermal, subcutaneous or intraarterial administration or in a form suitable for mucosal, in particular oral, intranasal or rectal administration.

The vaccine composition according to the invention may be provided in solution, as a suspension or as a solid preparation, e.g., in the form of a tablet or a suppository, in a prefilled syringe or as a spray.

The criteria for the suitability as a vaccine of the complex according to the invention are manifold. Thus, the immunogenicity of the complex may be used as a selection criterion. After administration to an animal, the respective immune reaction, e.g., the antibody titer may then be measured in the model and the amount or dose of vaccine required may be estimated.

If desired, protection may be another criterion for determining the suitability of the complex according to the invention for production of a vaccine. This may be an option if a challenge model for the respective pathogen is available. Another exposer of an animal model to the pathogen protection may be evaluated prior to the onset of disease or its consequences. The change in physiologic indicators such as antibody titer or other blood values, or death, may serve as outcome variables.

The vaccine is administered in an amount that has proved suitable in preclinical and clinical tests. For preclinical tests, the respective animal models are used.

The complex according to the invention may also be used for producing a preparation intended to immunize mammals or birds. Such a pharmaceutical preparation contains the complex according to the invention in an amount suitable for the immunization of mammals and birds to ensure adequate antibody formation.

After immunization, a polyspecific immunoglobulin preparation may, furthermore, be obtained from a body fluid of the mammal, e.g., from plasma, serum or colostrum, or from the bird's eggs. The polyspecific immunoglobulin preparation exhibits at least one specificity for the antigen and one specificity for the cell constituent and may be obtained from an appropriately immunized mammal or bird.

For producing the immunoglobulin preparation, the immunogen according to the invention is administered to a mammal, e.g., mouse, goat or rabbit at defined time intervals. After this, a body fluid such as blood or colostrum is taken from that mammal. From this, the immunoglobulin fraction is isolated by current methods. The antibodies contained preferably are directed against a plurality of protective antigens formed by a pathogen in the early phase of replication. According to another embodiment, antibodies that are directed against those protective antigens which are formed by the pathogen in the late phase of replication may also be obtained by the purposeful selection of antigens. Depending on the choice of antigen, the immunoglobulin preparation according to the invention also may contain antibodies against premature antigens or non-structural antigens. This is an advantage, in particular, in passive immunization with antibodies, since infected host cells, or host cells transformed by a recombinant gene or transfected host cells will be recognized by these antibodies and will be eliminated.

The invention also provides for a pharmaceutical preparation based on a nucleic acid and a cell constituent, which nucleic acid is capable of expressing an antigen forming a complex according to claim 1 with the cell constituent. The antigen is derived from a pathogen or is the pathogen in the inactive form. As already mentioned, the pathogen may be a virus, a bacterium a parasite, but also a molecular pathogen, e.g., a prion.

Both the nucleic acid and the cell constituent may be present nor only in isolated form, but also in purified form. The cell constituent may be provided also without additional purification, if care has been taken to avoid contamination, e.g., work was performed under sterile conditions.

The nucleic acid preferably is present as a plasmid having a promoter suitable for expressing the nucleic acid. For example, such plasmids may be commercially available plasmids. Promoters encompass current promoters such as a CMV or RSV promoter. In general, the choice of promoter is a function of the cell and the antigen to be expressed.

The nucleic acid, or expressed antigen, and the cell constituent preferably are present in an associated form. Association may be effected, for instance, via lipids or via a carrier, preferably an adjuvant. Association may be realized by covalent linkages, but also on grounds of nonspecific interactions, for instance due to electrostatic forces or van der Waals' forces.

Furthermore, a step for the inactivation of optionally present pathogens such as viruses is provided before or after the formation of the complex.

Using the complex according to the invention, a reagent suitable for detecting antibodies or recognizing an infection of a mammal with the pathogen may,also be produced. The reagent according to the invention, in particular, renders possible differentiation between infection with a pathogen and vaccination against the pathogen. Matveeva V. A. et al., 1995, Immunol. Lett. 46, 14, were able to demonstrate that sera of patients with flaviviruses infections contained antibodies that reacted with non structural proteins of the virus. After immunization with the whole-virus vaccine only antibodies against structural proteins of the virus can form. Differentiation is effected by obtaining a body fluid of the mammal, mixing the same with the reagent optionally immobilized on a carrier, and detecting by appropriate means the immunogen antibody reaction indicating an infection with the pathogen.

The invention also provides for a sets for recognizing an infection, of an organism with a pathogen. Such a set comprises the complex according to the invention and a means for detecting the specific reaction upon contact with the complex according to the invention such as, e.g., a complex/antibody reaction, which is indicative of an infection.

The means of detection is a labelled substrate such as antibodies specific for the antibody to be detected, e.g., IgM or IgG. Labelling is effected according to current methods, e.g. by means of a chromogen, fluorogen or radioactive substance.

The invention also contemplates processes for preparing the complex according to the invention. A first process for preparing the complex comprises the following steps:

obtaining an antigen from the pathogen or from the cell infected with the pathogen or by a chemical or biotechnological method,

obtaining the cell constituent of the mammalian cell,

linking the antigen to the cell constituent so as to obtain the complex,

isolating the complex, and

optionally purifying the complex.

Another process for preparing the complex according to the invention comprises the steps of:

infecting a mammalian cell with the pathogen,

optionally treating the cell with a view to releasing a cell fraction,

isolating the cell or cell fraction, respectively, containing the complex, and

optionally purifying the complex.

Yet another process for preparing the complex comprises the steps of:

obtaining a recombinant gene coding for the antigen,

transfecting or transforming a mammalian cell with the recombinant gene,

optionally treating the cell with a view to releasing a cell fraction,

isolating the cell or cell fraction, respectively, containing the complex, and

optionally purifying the complex.

According to a preferred embodiment, the mammalian cell or cell fraction, respectively, is enriched with at least one constituent according to any one of claims 9, 14 or 15.

The complex in a particularly preferred embodiment is a virus optionally in the inactive form, which is enriched with at least one constituent according to any one of claims 9, 14 or 15.

The following examples serve to elucidate the invention without limiting it thereto.

### EXAMPLE 1

Complex Based on a Cell Constituent of Virus-infected Cells

a) Cells

3T3 mouse fibroblasts (bred from BALB/c mice, haplotype H2^{d}) were cultivated in RPMI (GLBCO BRL, Gaithersburg, Md.) with 5% heat inactivated fetal calf serum (Hyclone Laboratories Logan, Utah), 2 mM glutamine, 100 μg/ml streptomycin and 100 U/ml penicillin G (all of JRH Biosciences, Lenexa, Kans.) at 37° C., 96% relative humidity and 5% CO₂.

b) Virus

Tick-borne encephalitis virus (TBE virus), strain Neudoerfl was purified by ultrafiltration (100 kd cut-off) and ultracentrifugation, from the supernatant of infected Vero cells according to WO 91/09935. For infection, the virus was appropriately diluted with sterile phosphate-buffered physiological saline (PBS).

c) Virus Titration

The titer of infectious TBE virus was identified by titration on PS cells, modified according to D E Madrid A T et al., Bull. World Health Organ., 1969, 40, 113-21. To this end, confluent single-cell layers of PS cells are incubated for one hour with a series of tenfold dilutions of a sample to be titrated, the sample is subsequently sucked off and replaced with a carboxymethylcellulose-containing medium. After four days, the cells were fixed with formaldehyde and stained with crystal violet. The holes formed in the cell bed, which are called plaques, are counted against a bright background, each hole corresponding to an infectious virus particle. Taking the dilution factor into account, the concentration of infectious virus particles contained in the sample is obtained, expressed in plaque forming Units (pfu) per ml.

d) Virus Inactivation, Cell Preparation

The cells were infected with the virus, the culture medium was sucked off the adherent cells, the cells were washed twice with 37° C. PBS and finally incubated for inactivation with a solution of 4% (w/v) paraformaldehyde in PBS for 10 minutes. The cells were washed once again, scraped off the surface of the cell culture by means of a cell scraper (Costar), suspended in PBS and centrifuged (1400 rpm-10 minutes-4° C.). The cell precipitate was resuspended, the cell concentration was determined in a counter chamber and the cell suspension was adjusted to the desired concentration.

e) Immunization

For immunization, the cells were adjusted to the concentrations indicated, either in 0.9% Al(OH)₃ or in PBS. Cells adjuvanted with alum were used for subcutaneous (sc) application, non-adjuvanted cells were injected intraperitoneally (ip), each at a volume of 0.2 ml per test animal. Test animals were female inbred BALB/c mice (haplotype H2^{d}) or C3H (haplotype H2^{k}) weighing between 15 and 17 g.

f) Immunity Test

After having completed the immunizations, the immunity of the animals to virus exposition was tested. To this end, immunized animals and an untreated control group were each injected i.p. with 1000 pfu per animal in a volume of 0.2 ml, which corresponded to approximately 100 times the lethal dose for 50% of the animals (LD₅₀).

The specificity of the humoral immunity for certain virus antigens was determined on a Western blot. Ad the antigen preparation, infected or non-injected 3T3 fibroblasts were dissolved in a lysis buffer (20 mM Tris, 150 mM NaCl, 1% NP-40 detergent, pH 7.5 with the protease inhibitors phenyl-methyl-sulfonyl fluoride [2 mM], aprotinin and leupeptin [10 μg/ml each], and the clear supernatant obtained by centrifugation, i.e., a complex mixture of virus and cell proteins, was electrophoretically separated on polyacrylamide gel (cf. Laemmli U K, Nature, 1970, 227, 680-5). The separated proteins were then electrophoretically transferred to a nitrocellulose membrane (cf. Towbin H., et al., Proc. Natl. Acad., Sci., USA, 1979, 76 (4350-4). After having blocked additional protein binding sites with the aid of a milk powder solution (1%), the membrane thus obtained was further incubated with sera of immunized or control animals, bound antibodies were bound against mice immunoglobulin with the aid of a horseradish peroxidase-coupled second antibody, and that bond was detected by means of enhanced chemilunminescence (Amersham). By comparison with a molecular weight marker, the magnitude of the detected protein bands was determined.

g) Results and Discussion

The immunization of mice according to the procedure described induced a protective immune response against subsequent infection with a 100-fold LD₅₀ dose of TBE virus. Protection was obtained with some 5×10⁷ cells after a three-time immunization of the animals at intervals of 2 weeks each. No relevant difference was to be noted between subcutaneous application in alum or intraperitoneal application without adjuvant.

In respect of the immunization of animals wit homologous (histoidentical) or heterologous (histoincompaiible) cells, it was interesting to note that homologous immunization apparently induced better protection than heterologous immunization. Although it has so far been possible to demonstrate passive protection by the transfer of TBE virus antibodies (cf. Heinz F X et al., Virology, 1983, 126, 525-37) or antisera (cf. Heinz F X et al., Infect. Immun., 1981, 33, 250-7), it is to be assumed in the light of these experimental data that also cell-mediated immunity contributes its share to protection in the experimental system described.

It could be demonstrated in the Western blot that both paths of immunization resulted in humoral immunity, since sera of both groups exhibited clear reactivities with virus-encoded proteins. In this context, it is still particularly noteworthy that not only the viral surface protein glyco-E, but also at least one non-structural protein of the virus was recognized, e.g., NS1. That protein, although coded by the TBE virus genome, is not present in the mature virion. The protein is, however, known to be expressed on the surface of flaviviruses-infected cells, in particular cells infected with TBE, and immunization with NS1 of flaviviruses may induce protective immunity. While with conventional immunizations with an inactivated virus such non-structural determinants are not offered to the immune system, immunity against both non-structural and structural proteins of a virus may be induced by the path described herein. Even when administering suboptimal amounts of TBE virus to the animals, a clearly extended mean survival time could be observed.

### EXAMPLE 2

Complex Based on Transfected Cells

3T3 cells (3T3, H-2D^{d}, ATCC CCL 163) or 3T3 cells transfected with the gp160-encoding gene of the Human Immunodeficiency Virus Type I (3T3-gp160, prepared according to Felgner et al., 1987, Proc. Natl. Acad. Sci., USA, 84, 7413) were used as an immunogen. Prior to immunization the cells were irradiated with 50 Gy of a ¹³⁷Cs source. Thereafter, BALB/c mice (Charles River, Sulzfeld, Germany) were intraperitoneally immunized with 5×10⁶ 3T3 or 3T3-gp160. That immunization was repeated twice at three-week intervals. One week after the final immunization, blood was collected from the mice in order to determine the cellular immunity of the spleen as well as the humoral immunity.

a) Cellular Immunity

The ability of the complex according to the invention to induce cellular immunity was determined by way of T cell-mediated cytotoxicity detection. To this end, a single-cell suspension of the immunocompetent cells was recovered from the spleens by careful passage through a wire mesh screen, filtration through sterile cotton and subsequent hemolysis of the erythrocytes. These cells were then resuspended in complete culture medium consisting of RPMI 1640 (Flow Laboratories, Irvine, UK) supplemented with 10% heat inactivated fetal calf serum (JRH Biosciences, Lenexa, Kans.), 2 mM L-glutamine (Gibco, Paisley, Scotland), 100 IU/ml penicillin and 100 μg/ml streptomycin (both from Gibco) as well as 5×10⁻⁵ M 2-mercaptoethanol (Biorad, Hercules, Calif., USA). For in vitro restimulation of the cytotoxic T cells, the immunocompatible spleen cells were seeded at a density of 5×10⁶ cells per culture well of a 24-well culture plate (Costar) and stimulated by the addition of 0.15 μM gp120 peptide 312-327 (Neosystems, Strasbourg, France).

After a 5-day incubation at 37° C. and 5% CO₂ the spleen cells were again harvested and the dead cells eliminated by centrifugation over a lympholyte-M gradient (Cedarlane, Ontario, Canada). The live spleen cells were then used as effector cells for determining the cell-mediated cyrotoxicity in a ⁵¹Cr release test. To this end, 3T3 and 3T3-gp160 cells were at first radioactively labelled with 14.8 MBq Na₂⁵¹CrO₄ (Amersham, Amersham, Buckinghamshire, UK) for two hours as target cells. After this, 1×10⁴ of these target cells were each pipetted into two culture wells of a 96-well V-bottom microtiter culture plate (Nunc, Roskilde, Denmark) and the effector cells were added at the indicated ratios. The plates were then incubated at 37° C. for 4 hours and at the end of the incubation time 100 μl supernatant were taken from each culture well. The radioactivity contained therein and released by a possible lysis of the target cells was measured in a y-radiation counter (Packard, Meidan, Conn.). The spontaneous and maximum ⁵¹Cr release was determined by incubation of the target cells with culture medium alone and with a solution of 1% Triton X-100. The percentage of the specific lysis was calculated using the formula:

(cpm test release−cpm spontaneous release)/(cpm maximum release−cpm spontaneous release).

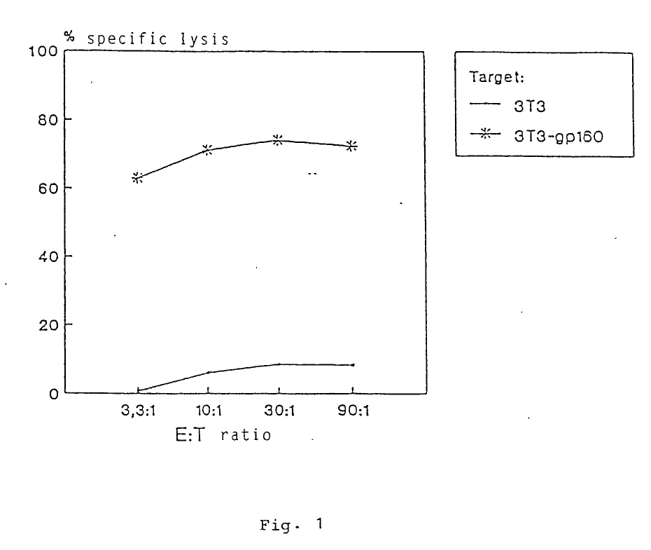

FIG. 1 indicates the percentage of the specific lysis by effector cells from mice immunized with gp160 IIIB transfected 3T3. While these effector cells lysed the transfected target cells by up to 79%, the non-transfected 3T3 were only slightly lysed.

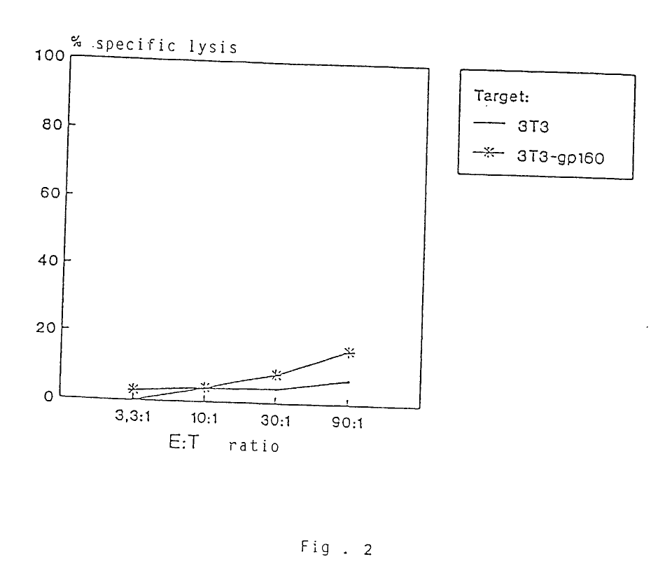

By contrast, FIG. 2 indicates the percentage of the specific lysis by effector cells from mice immunized with non-transfected 3T3. These effector cells caused no remarkable lysis of the target cells. These results have thus demonstrated that the construct according to the invention is able to stimulate an HIV-1-specific cellular immune response and, in particular, cytotoxic T-cells directed against the HIV-1 envelope protein gp160.

b) Humoral Immunity

In order to determine the humoral immune response, blood was collected from the immunized mice under slight ether anesthesia. From this, serum was obtained which was assayed for anti-gp 120 IIIB-IgG antibodies by an enzyme immunoassay. To this end, the culture wells of a 96-well plate (flat bottom, high binding, FA Costar, Cambridge, Mass., USA) were each filled with 100 μl of a solution of gp120 (HIV-1 IIIB, Intracell, London) at a concentration of 1 μg/ml and incubated at 37° C. for one hour. After that layering of the culture wells with the gp120 protein, the plate was washed and remaining free binding sites in the culture wells were saturated with 250 μl 2% BSA (bovine serum albumin) in PBS (phosphate-buffered saline). The serum samples to be tested, and a control serum that was gp160 antibody-positive, were applied in various dilution steps in 100 μl per culture well. After incubation for 16 hours at 37° C. the plate was washed anew. For detecting the bound gp120-specific antibodies, a peroxidase-labelled goat anti-mouse-IgG antibody solution (Accurate Chem., Westbury, N.Y., USA) in a 1:50 000 dilution was added for one hour. Thereafter, the plate was washed again and an OPD substrate solution was added (ortho-phenylene diamine, 3 mg/ml, Sigma, St. Louis, Mo., USA). The enzyme reaction was stopped after 30 minutes with a 2N sulfuric acid solution and the dye formed was measured with a Nunc immunoreader at 490 nm (reference filter 620 nm). The titer of the assayed sample resulted from the reciprocal value of the highest sample dilution whose optical density had a value greater than OD 0.2.

In Table 1 the serum titer of gp160 IIIB-specific IgG of the mice immunized as described above is listed under the heading ELISA. From these values it is apparent that the construct according to the invention is able to induce also a humoral immune response. The values indicated under the heading cytotoxicity once more indicate the induction of a cellular immune response.

### EXAMPLE 3

Complex Based on Transfected Cells and Comprising an Adjuvant

gp160 IIIB-transfected 3T3 cells as described in Example 1 and additionally mixed either with PBS or with 5 μg/ml cholera toxin B (CTB, List Biologicals, Campbell, Calif., USA) were used as the immunogen. BALB/c mice were then intubated by means of a feeding needle (Nordland, Hamburg, Germany) and 5×10⁶ cells of the immunogen were administered directly into the stomach. Two hours before, the mice had been fasted and 30 minutes before immunization the gastric acid of the mice was neutralized by administering 1.5% NaH₂CO₃ solution. Mucosal immunization was repeated twice at one-week intervals. One week after the last immunization, cellular immunity was determined as described in Example 1.

TABLE 1

| | | | | In vitro test | |
|---|---|---|---|---|---|
| | | | | Cytotoxicity | ELISA |
| Immunization | | | | gp160 IIIB | gp160 IIIB |
| | | | Interval | spec. lysis* | spec. IgG |
| Immunogen | Route | Number | weeks | % | Titer |
| 3T3 | i.p. | 3x | 3 | 8.7 | 200 |
| 3T3-gp160 | i.p. | 3x | 3 | 64.0 | 12800 |
| 3T3-gp160 | mucosal | 3x | 1 | 40.0 | — |

*Effector: target ratio = 90:1

From Table 2 it is apparent that HIV-1-specific cell-mediated immunity could be induced also by mucosal immunization with gp160-transfected cells (in the presence of an appropriate adjuvant).

### EXAMPLE 4

Induction of an Immune Response by Infected Cells

3T3 cells or Vero cells (ATCC CCL81) were infected with the recombinant vaccinia virus vSC25 (provided by Dr. Barret, Immuno AG, Orth, Austria) containing the genetic information for gp160 IIIB for 16 hours at a multiplicity of infection of 5. Thereafter, BALB/c mice were each mucosally immunized with 5×10⁶ of these cells or non-infected control cells using a feeding needle. Immunization was repeated twice at three-week intervals. One week after the last immunization cellular immunity was determined as described in Example 1. Table 2 shows that also mucosal immunization with recombinant vaccinia virus-infected cells induced a HIV-1-specific cytotoxic T cell response. The immune-response was independent of the cell type used for immunization.

TABLE 2

| | | | | In vitro test |
|---|---|---|---|---|
| | | | | Cytotoxicity |
| Immunization | | | | gp160 IIIB |
| | | | Interval | spec. lysis* |
| Immunogen | Route | Number | weeks | % |
| 3T3 | mucosal | 3x | 3 | 18.4 |
| 3T3-vSC25 | mucosal | 3x | 3 | 68.9 |
| Vero | mucosal | 3x | 3 | 6.6 |
| Vero-vSC25 | mucosal | 3x | 3 | 63.6 |

*Effector: target ratio = 90:1

1. A complex comprising an antigen of a microbial or molecular pathogen and cell constituents of mammalian cells in the isolated or purified form.

2. A complex according to claim 1, characterized in that the antigen is derived from a virus, bacterium, parasite or a prion.

3. A complex according to claim 1 or 2, characterized in that the antigen is an inactive virus.

4. A complex according to claim 3, characterized in that the virus is an,inactivated virus and, in particular, a virus inactivated by a chemical and/or physical process.

5. A complex according to claims 1 to 4, characterized in that the antigen is encoded by a recombinant nucleic acid.

6. A complex according to any one of claims 1 to 5, characterized in that the cell constituent is derived from infected cells.

7. A complex according to any one of claims 1 to 5, characterized in that the cell constituent is derived from a cell containing a recombinant gene.

8. A complex according to any one of claims 1 to 5, characterized in that the cell constituent is derived from an activated cell.

9. A complex according to any one of claims 1 to 5, characterized in that the cell constituent is derived from cells originating from the species for which the virus is pathogenic.

10. A complex according to any one of claims 1 to 9, characterized in that the cell constituent is contained in a fraction containing the cell membrane or an intracellular structure of the cell.

11. A complex according to any one of claims 1 to 10, characterized in that the virus is a human pathogenic virus and the cell constituent is derived from human cells.

12. A complex according to any one of claims 1 to 11, characterized in that the complex linkage is a covalent linkage, optionally by means of a linker.

13. A complex according to any one of claims 1 to 12, characterized in that the complex linkage is effected by electrostatic, hydrophobic or van der Waals' forces.

14. A complex according to any one of claims 1 to 13, characterized in that a substance selected from the group consisting of membrane protein, intracellular protein, factor derived from activated lymphocytes and activator for lymphocytes or a combination thereof is contained in the cell fraction.

15. A complex according to any one of claims 1 to 14, characterized in that the membrane protein and/or the intracellular protein is an alloantigen chemokine receptor, chemokine or lymphokine or a combination thereof.

16. A complex according to any one of claims 1 to 15, characterized in that the complex is comprised of an inactive virus according to claim 1 having a cell constituent according to any one of claims 9, 14 or 15 incorporated therein.

17. A complex according to any one of claims 1 to 16, characterized in that the antigen and the cell constituent are adsorbed on a solid carrier, in particular a lipid component.

18. A complex according to any one of claims 1 to 17, characterized in that the complex is obtainable from a cell infected with the pathogen.

19. A complex according to any one of claims 1 to 18, characterized in that the complex is obtainable from a cell containing a recombinant gene.

20. A vaccine composition containing the complex according to any one of claims 1 to 19, where the vaccine composition is presented in a pharmaceutically acceptable form for administration in an amount suitable for the prophylactic or therapeutic treatment of disease caused by the pathogen in mammals and, in particular, primates.

21. A vaccine composition according to claim 20, characterized in that the vaccine composition contains additional antigens or adjuvants.

22. A pharmaceutical preparation containing the complex according to any one of claims 1 to 19 in an amount, suitable for the immunization of mammals or birds.

23. A pharmaceutical preparation based on a nucleic acid and a cell constituent, which nucleic acid is capable of expressing an antigen forcing a complex according to claim 1 with the cell constituent.

24. A preparation according to claim 23, characterized in that the nucleic acid is present as a plasmid comprising a promoter suitable for the expression of the nucleic acid.

25. A preparation according to claim 3 or 24, characterized in that the antigen has the structure of a pathogen, yet is not pathogenic.

26. A preparation according to any one of claims 23 to 25, characterized in that the nucleic acid, or the expressed antigen, and the cell constituent are present in a form associated with each other via lipids or a carrier, preferably via an adjuvant.

27. A preparation according to any one of claims 23 to 26, characterized in that the nucleic acid, or the expressed antigen, and the cell constituent are present in a form associated via van der Waals' or electrostatic interactions.

28. A polyspecific immunoglobulin preparation exhibiting a specificity for an antigen originating from a microbial or molecular pathogen and a specificity for a cell constituent of a mammalian cell, which is obtainable by immunizing a mammal or a bird with a pharmaceutical preparation according to claim 15 and recovering the immunoglobulin preparation from a body fluid of the mammal or from the bird's eggs, respectively.

29. A process for preparing the complex according to any one of claims 1 to 19, comprising the following steps:
obtaining an antigen from the pathogen or from the cell infected with the pathogen or by a chemical or biotechnological method,
obtaining The cell constituent of the mammalian cell,
linking the antigen to the cell constituent so as to obtain the complex,
isolating the complex, and
optionally putting the complex.

30. A process for preparing the complex according to any one of claims 1 to 19, comprising the following steps:
infecting a mammalian cell with the pathogen,
optionally treating the cell to induce it to release a cell fraction,
isolating the cell or cell fraction, respectively, containing the complex, and
optionally purifying the complex.

31. A process for preparing the complex according to any one of claims 1 to 19, comprising the following steps:
obtaining a recombinant gene coding for the antigen,
transfecting or transforming a mammalian cell with the recombinant gene,
optionally treating the cell with a view to releasing a cell fraction,
isolating the cell or cell fraction, respectively, containing the complex, and
optionally purifying the complex.

32. A process according to any one of claims 29 to 31, characterized in that the mammalian cell, or the cell fraction, is enriched with at least one of the constituents according to any one of claims 9, 14 or 15.

33. A process according to claim 30 or 32, characterized in that the complex is a virus, optionally in the inactive form, that is enriched with at least one of the constituents according to any one of claims 9, 14 or 15.
